(19) United States
(12) Patent Application Publication
(10) Pub. No.: US 2004/0014054 A1
(43) Pub. Date: Jan. 22, 2004

(54) Biosensor chip

(76) Inventors: Alexander Frey, Munchen (DE); Roland Thewes, Grobenzell (DE)

Correspondence Address: Jeffrey R Stone, Briggs & Morgan, 2400 Ids Center, 80 South Eigth Street, Minneapolis, MN, 55402, US

(21) Appl. No.: 10/239,622

(22) PCT Filed: Mar. 29, 2001
(86) PCT No.: PCT/DE01/01241

(30) Foreign Application Priority Data
Mar. 30, 2000 (DE) 10015816.1

(51) Int. Cl. C12Q 1/68; C12M 1/34

(52) U.S. Cl. 435/6; 435/287.2; 205/777.5

(57) ABSTRACT

The invention relates to a biosensor chip that is provided with a first electrode and a second electrode. The first electrode is provided with a holding area for holding probe molecules which can bind macromolecular biopolymers. The invention also relates to an integrated electric differentiating circuit by means of which an electric current can be detected and can be differentiated according to time, whereby said current is generated during a reduction/oxidation recycling procedure.

A biosensor chip of this type is known from [1].

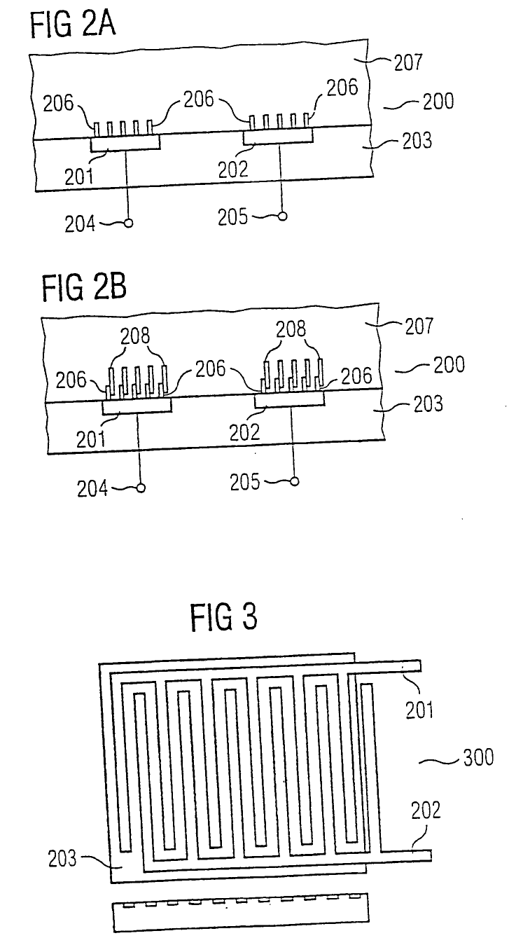

FIG. 2a and FIG. 2b show such a biosensor chip, as described in [1]. The sensor 200 has two electrodes 201, 202 made of gold, which are embedded in an insulator layer 203 made of insulator material. Electrode terminals 204, 205, to which the electrical potential applied to the electrode 201, 202 can be delivered, are connected to the electrodes 201, 202. The electrodes 201, 202 are arranged as planar electrodes. DNA probe molecules 206 are immobilized on each electrode 201, 202 (cf. FIG. 2a). The immobilization is carried out according to the gold-sulfur coupling. The analyte to be studied, for example an electrolyte 207, is applied to the electrodes 201, 202.

If DNA strands 208 with a sequence which is complementary to the sequence of the DNA probe molecules 206 are contained in the electrolyte 207, then these DNA strands 208 hybridize with the DNA probe molecules 206 (cf. FIG. 2b).

Hybridization of a DNA probe molecule 206 and a DNA strand 208 takes place only if the sequences of the respective DNA probe molecule 206 and of the corresponding DNA strand 208 are complementary to one another. If this is not the case, then no hybridization takes place. A DNA probe molecule with a predetermined sequence is hence respectively able to bind only a particular DNA strand, namely the one with the complementary sequence in each case, i.e. to hybridize with it.

If hybridization takes place, then as can be seen from FIG. 2b, the value of the impedance between the electrodes 201, 202 becomes modified. This modified impedance is determined by applying an AC voltage with an amplitude of approximately 50 mV to the electrode terminals 204, 205 and determining the resulting current by means of a connected measuring instrument (not shown).

In the event of hybridization, the capacitive component of the impedance between the electrodes 201, 202 is reduced. This is attributable to the fact that both the DNA probe molecules 206 and the DNA strands 208, which may hybridize with the DNA probe molecules 206 if appropriate, are non-conductive and therefore clearly shield the respective electrode 201, 202 electrically to a certain extent.

In order to improve the measurement accuracy, it is also known from [4] to use a plurality of electrode pairs 201, 202 and to connect them in parallel, these being clearly arranged interdigitated with one another, so that a so-called interdigitated electrode 300 is obtained. The dimensioning of the electrodes and the distances between the electrodes are of the order of the length of the molecules to be detected, i.e. the DNA strands 208 or less, for example in the region of 200 nm and less.

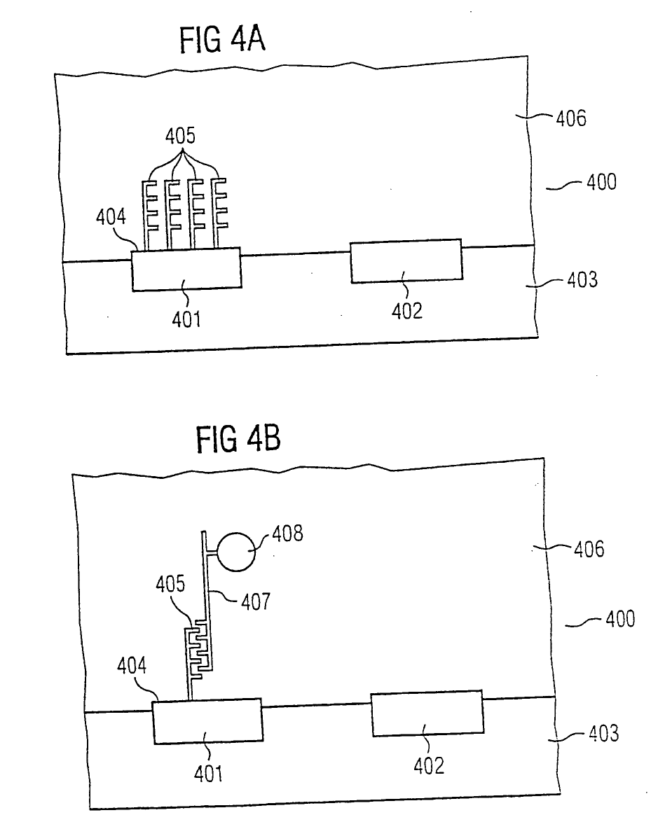

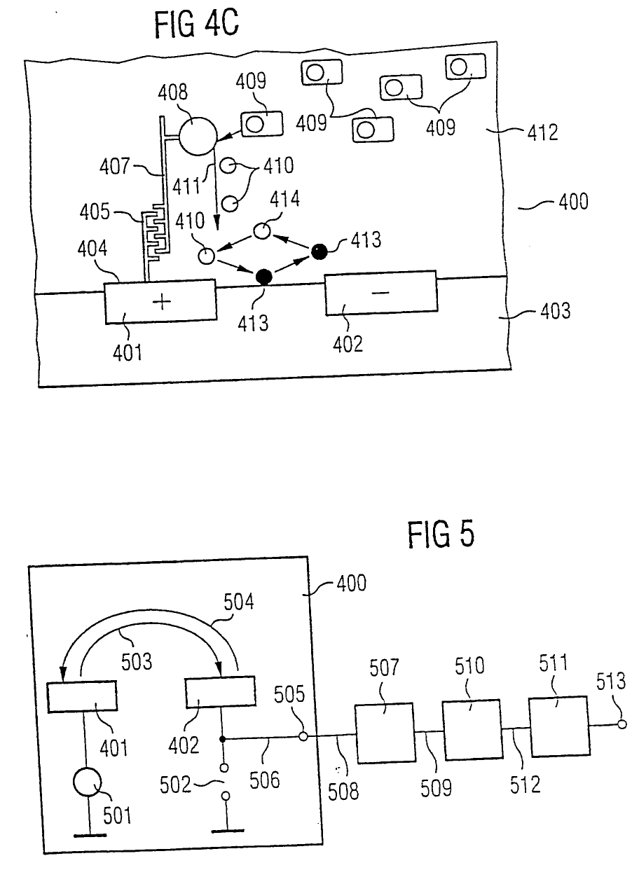

Furthermore, basic principles concerning a reduction/oxidation recycling operation for recording macromolecular biopolymers are known from [2] and [3]. The reduction/oxidation recycling operation, which is also referred to below as the redox recycling operation, is explained in more detail below with reference to FIG. 4a to FIG. 4c.

FIG. 4a shows a biosensor chip 400 having a first electrode 401 and a second electrode 402, which are applied to a substrate 403 as insulator layer.

A holding region, configured as holding layer 404, is applied to the first electrode 401 made from gold. The holding region is used to immobilize DNA probe molecules 405 on the first electrode 401.

There is no such holding region on the second electrode.

If DNA strands with a sequence which is complementary to the sequence of the immobilized DNA probe molecules 405 are to be recorded by means of the biosensor 400, the sensor 400 is brought into contact with a solution 406 which is to be analyzed, for example an electrolyte, in such a manner that any DNA strands which are present in the solution 406 which is to be analyzed and have the complementary sequence to the sequence of the DNA probe molecules 405 can hybridize.

FIG. 4b shows the situation in which the DNA strands 407 which are to be recorded are present in the solution 406 which is to be analyzed and have hybridized with the DNA probe molecules 405.

The DNA strands 407 in the solution which is to be analyzed are marked with an enzyme 408, allowing molecules which are described below to be cleaved into part-molecules.

There is usually a considerably greater number of DNA probe molecules 405 than the number of DNA strands 407 to be determined which are present in the solution 406 which is to be analyzed.

After the DNA strands 407 which may be present in the solution 406 which is to be analyzed, having the enzyme 408, have hybridized with the immobilized DNA probe molecules 407, the biosensor chip 400 is rinsed, with the result that the unhybridized DNA strands are removed and the solution 406 which is to be analyzed is cleaned off the biosensor chip 400.

An electrically uncharged substance which contains molecules which can be cleaved by the enzyme at the hybridized DNA strands 407 into a first part-molecule 410 with a negative electric charge and into a second part-molecule with a positive electric charge is added to this rinsing solution which is used for rinsing or to a further solution which is supplied specifically for this purpose in a further phase.

As shown in FIG. 4c, the negatively charged first part-molecules 410 are attracted to the positively charged anode, i.e. to the first electrode 401, as indicated by the arrow 411 in FIG. 4c. The negatively charged first part-molecules 410 are oxidized at the first electrode 401, which as anode has a positive electrical potential, and as oxidized part-molecules 413 are attracted to the negatively charged cathode, i.e. the second electrode 402, where they are reduced again. The reduced part-molecules 414 in turn migrate to the first electrode 401, i.e. to the anode.

In this way, an electric circuit current is generated, which is proportional to the number of charge carriers which are in each case generated by the enzymes 408.

The electrical parameter which is evaluated in this method is the change in the electric current

$$\frac{ⅆ I}{ⅆ t}$$

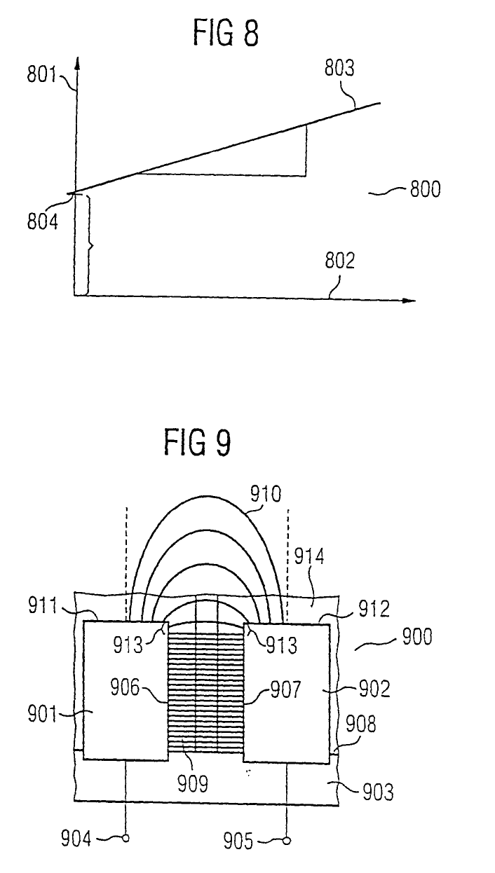

as a function of time t, as diagrammatically illustrated in diagram 800 in FIG. 8.

FIG. 8 shows the function of the electric current I 801 as a function of time t 802. The resulting curve 803 has an offset current I_{offset} 804 which is independent of the time profile.

The offset current I_{offset} 804 is generated by parasitic components on account of imperfections in the biosensor chip 400.

A significant cause of the offset current I_{offset} 804 is that the coverage of the first electrode 401 with DNA probe molecules 405 is not ideal, i.e. is not completely dense. In the case of a completely dense coverage of the first electrode 401 with DNA probe molecules 405, only purely capacitive electrical coupling would result on account of what is known as the double-layer capacitance formed by the immobilized DNA probe molecules 405 between the first electrode 401 and the electrically conductive electrolyte 406.

However, the incomplete coverage leads to parasitic current paths between the first electrode 401 and the solution 406 which is to be analyzed, and these paths have, inter alia, ohmic components.

In order, however, to allow the oxidation/reduction process to take place, the coverage of the first electrode 401 with the DNA probe molecules 405 must not be complete, so that the electrically charged part-molecules, i.e. the negatively charged first part-molecules are attracted to the first electrode 401 at all.

On the other hand, to achieve the highest possible sensitivity of a biosensor of this type, combined with low parasitic effects, the coverage of the first electrode 401 with DNA probe molecules 405 should be as dense as possible.

To achieve a high reproducibility of the measured values determined using a biosensor 400 of this type, both electrodes 401, 402 must always provide a sufficiently large surface area for the oxidation/reduction process as part of the redox recycling operation.

FIG. 5 shows a sketch of the biosensor chip 400 in accordance with the prior art and the metrological determination of the parameter.

$$\frac{ⅆ I}{ⅆ t} .$$

To simplify explanation, FIG. 5 symbolically depicts a first voltage source 501, which provides a first electrical potential V1 of the first electrode 401, and a second voltage source 502, which provides a second electrical potential V2 of the second electrode 402.

Furthermore, two arrows 503, 504 symbolically depict the electric circuit current which is established in accordance with the redox recycling operation, as explained above.

The resulting measurement current I, the profile of which over the course of time is given in accordance with the following rule:

I=I_{offset}+m·t, (1)

where

$$m = \frac{ⅆ I}{ⅆ t} , \qquad ( 2 )$$

where

I is the value of the measurement current recorded at a given time,

I_{offset} is the offset current,

$$\frac{ⅆ I}{ⅆ t}$$

is the differentiation of the circuit current after time t, and

t is the time,

is present at an external electrical connection 505 of the biosensor chip 400, which is coupled to the second electrode 402 via an electrical line 506, and can be tapped externally from the biosensor chip 400.

An electrical measuring appliance 507 is coupled to the biosensor chip 400 via an electrical line 508. The measuring appliance 507 is coupled to an electronic memory 510 for storing the tapped values of the measurement current I at different times at the electrical connection 505 of the biosensor chip 400 via a further electrical line 509, for example an electrical cable.

Furthermore, an evaluation unit 511 is coupled to the memory 510 via an electrical line 512. The electrical measurement currents I which were recorded at various times and were stored in the memory 510 are read in the evaluation unit 511, and the increase m in the curve profile 503 of the recorded measurement current I over the course of time t is determined. Clearly, numerical differentiation of the recorded circuit current is carried out in the evaluation unit 511.

The value m which then results is made available at an output 513 of the evaluation unit 511.

Known methods can then be used to work out the number of hybridized DNA strands marked with the enzyme 408 on the first electrode 401 on the basis of the parameter m.

In this context, it should be noted that the offset current I_{offset} is usually very much greater than the change in the circuit current over the course of time, i.e. the following relationship applies:

I_{offset}>>m·tₘₑₛₛ, (3)

where tₘₑₛₛ denotes the total measurement time during which the circuit current is determined by means of the biosensor chip 400.

Therefore, in the context of a current signal with a high absolute value, i.e. the offset current I_{offset}, the biosensor chip 400 has to measure what is in relative terms a very small, time-dependent change with a high level of accuracy.

Consequently, high demands are imposed on the measuring appliance 507 which is to be used.

Furthermore, a fundamental problem is that, on account of the relationship presented above, the method is highly sensitive to signal noise.

Even interference of the order of magnitude of

$$\frac{m \cdot {t}_{mess}}{{I}_{offest}} \qquad ( 4 )$$

which, as stated above, may be very minor, can lead to loss of the information, i.e. to incorrect evaluation and therefore to an incorrect measurement result.

Therefore, the invention is based on the problem of describing a biosensor chip by means of which the increase in the profile of the circuit current over the course of time within the context of the redox recycling operation can be recorded with increased reliability.

The problem is solved by the biosensor chip having the features described in the independent patent claim.

A biosensor chip has a first electrode and a second electrode. The first electrode has a holding region for holding probe molecules which can bind macromolecular biopolymers. The first electrode and the second an electrode are designed in such a manner that a reduction/oxidation recycling operation can take place at them. Furthermore, an integrated electrical differentiator circuit, which can be used to record an electric current generated during the reduction/oxidation recycling operation and to differentiate it over the course of time, is integrated in the biosensor chip.

The term macromolecular biopolymers is to be understood as meaning, for example, proteins or peptides or also DNA strands of in each case a predetermined sequence.

Irrespective of which type of macromolecular biopolymer is to be recorded in the solution which is to be analyzed, the macromolecular biopolymer can be marked in advance with the enzyme.

If proteins or peptides are to be recorded as macromolecular biopolymers, the immobilized molecules are ligands, for example active substances with a possible binding activity which bind the proteins or peptides which are to be recorded to the respective electrode on which the corresponding ligands are arranged.

Suitable ligands are enzyme agonists or enzyme antagonists, pharmaceuticals, sugars or antibodies or any molecule which has the ability to specifically bind proteins or peptides.

If DNA strands of a predetermined sequence are used as macromolecular biopolymers which are to be recorded by means of the biosensor, the biosensor can be used to hybridize DNA strands of a predetermined sequence to DNA probe molecules which have a complementary sequence to the sequence of the DNA strands, as molecules on the first electrode.

In the context of the present description, the term probe molecule is to be understood as meaning both a ligand and a DNA probe molecule.

The holding region may be designed to hold probe molecules to which peptides or proteins can be bound.

Alternatively, the holding region may be designed to hold DNA probe molecules to which DNA molecules can be bound.

The holding region may include at least one of the following materials:

hydroxyl radicals, epoxy radicals, amine radicals, acetoxy radicals, isocyanate radicals, succinimidyl ester radicals, thiol radicals, gold, silver, platinum, titanium.

The biosensor chip may be provided with a third electrode, in which case the second and third electrodes are designed in such a manner that the reduction/oxidation process as part of the reduction/oxidation recycling operation takes place at the second electrode and at the third electrode.

In this context, the first electrode may have a first electrical potential, the second electrode may have a second electrical potential, and the third electrode may have a third electrical potential. The third electrical potential is selected in such a manner that during the reduction/oxidation recycling operation the reduction or oxidation takes place only at the second electrode and at the third electrode.

This can be ensured, for example, by the third electrical potential being greater than the first electrical potential and the first electrical potential being greater than the second electrical potential.

According to a refinement of the invention, the electrodes may be arranged in an interdigitated electrode arrangement, the third electrode in each case being arranged between the first electrode and the second electrode.

Furthermore, the first electrode and the second electrode and/or the third electrode may be arranged in such a manner relative to one another that substantially uncurved field lines of an electric field, which is generated between the first electrode and the second electrode and/or the third electrode, can form between the first electrode and the second electrode and/or the third electrode.

According to a further configuration of the invention, the differentiator circuit is electrically coupled to the second electrode. The differentiator circuit may be coupled to the second electrode via a current-voltage converter.

Furthermore, a reference circuit, which has the same structure as the differentiator circuit, if appropriate with the current-voltage converter, may be integrated on the biosensor chip. The reference circuit can be used to generate an electrical reference signal.

The reference circuit can be used to carry out automatic calibration of fluctuations in the units which determine the functionality and dimensioning of the differentiator circuit, in particular the electrical resistances and the capacitance, which may be considerable for different chips and wafers. In this way, the quality of the measurement result achieved is further increased.

To filter out the noise signal, a low-pass filter may be provided in the reference circuit, the cut-off frequency of the low-pass filter being such that although the high-frequency noise signal is filtered out, nevertheless the corresponding change in the recorded circuit current over the course of time can be taken into account in the context of the differentiator circuit.

A further increase in the robustness of the measurement result determined is achieved by the frequency band limitation in the reference circuit by means of the low-pass.

Furthermore, the biosensor chip may have a multiplicity of first electrodes and a multiplicity of second electrodes, the first and second electrodes being arranged as an electrode array within the biosensor chip.

Furthermore, a multiplicity of third electrodes can be provided and arranged as an electrode array, the second electrodes and the third electrodes being designed and arranged in such a manner that the reduction/oxidation process takes place as part of a reduction/oxidation recycling operation at the second electrodes and at the third electrodes.

Clearly, the invention can be considered to lie in the fact that the differentiation of the circuit current determined is no longer recorded outside the chip, but rather on-chip recording of the resulting circuit current and/or its profile over the course of time can now be recorded with greater robustness compared to the biosensor chip according to the prior art.

If large numbers of DNA strands which are marked with the enzyme hybridize with the immobilized DNA probe molecules within a small area, a correspondingly large number of these enzymes is concentrated at this area, and the rate at which the circuit current generated rises is higher than in a different area, where fewer DNA strands marked with the enzyme are hybridized. By comparing the rates of increase between various areas of the biosensor, it is possible to determine not only whether DNA strands in the solution which is to be analyzed hybridize with the DNA probe molecules of a predetermined sequence, but also how well, i.e. with what efficiency, the hybridization takes place compared with other DNA probe molecules.

In other words, this means that a biosensor chip of this type provides qualitative and quantitative information about the DNA content of a solution which is to be analyzed.

Exemplary embodiments of the invention are illustrated in the figures and explained in more detail below.

In the drawing:

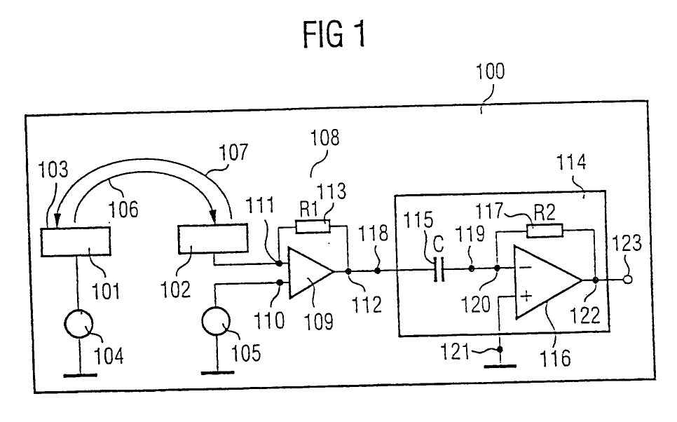

FIG. 1 shows a sketch of a biosensor chip in accordance with an exemplary embodiment of the invention;

FIGS. 2a and 2b show a sketch of two planar electrodes, by means of which the existence of DNA strands which are to be recorded in a solution which is to be analyzed (FIG. 2a) or their nonexistence (FIG. 2b) can be detected;

FIG. 3 shows interdigitated electrodes according to the prior art.

FIGS. 4a to 4c show sketches of a biosensor chip in accordance with the prior art, on the basis of which individual states as part of the redox recycling operation are explained;

FIG. 5 shows a sketch illustrating the evaluation of the measurement current in accordance with the prior art;

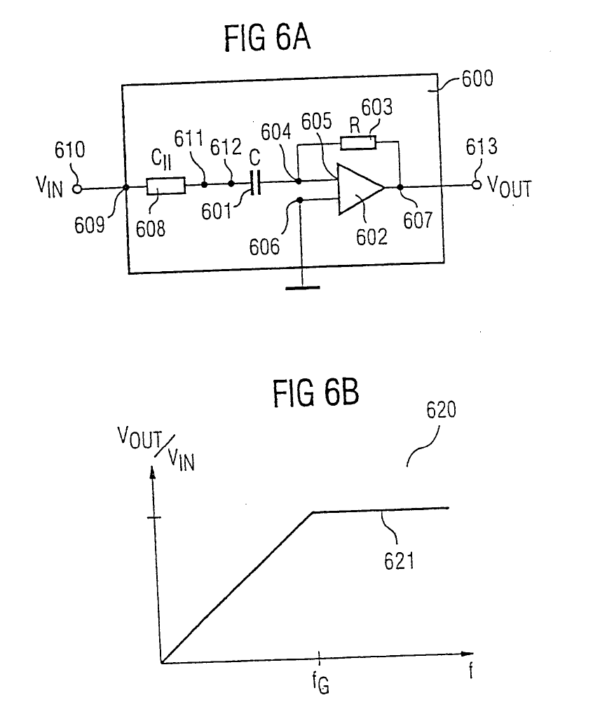

FIGS. 6a and 6b show a sketch of the reference circuit (FIG. 6a) with band limitation and a Bode diagram which shows the band limitation in accordance with an exemplary embodiment of the invention (FIG. 6b); and

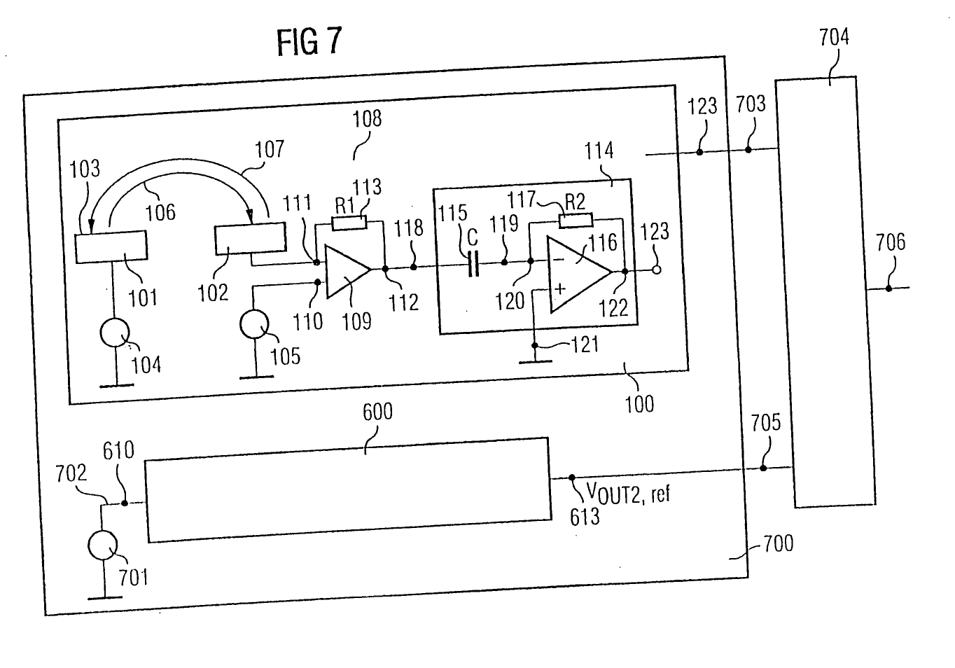

FIG. 7 shows a sketch of the biosensor chip in accordance with a further exemplary embodiment of the invention with an integrated reference circuit.

FIG. 8 shows a functional curve of a circuit current in accordance with the prior art as part of a redox recycling operation;

FIG. 9 shows a biosensor in accordance with an exemplary embodiment of the invention;

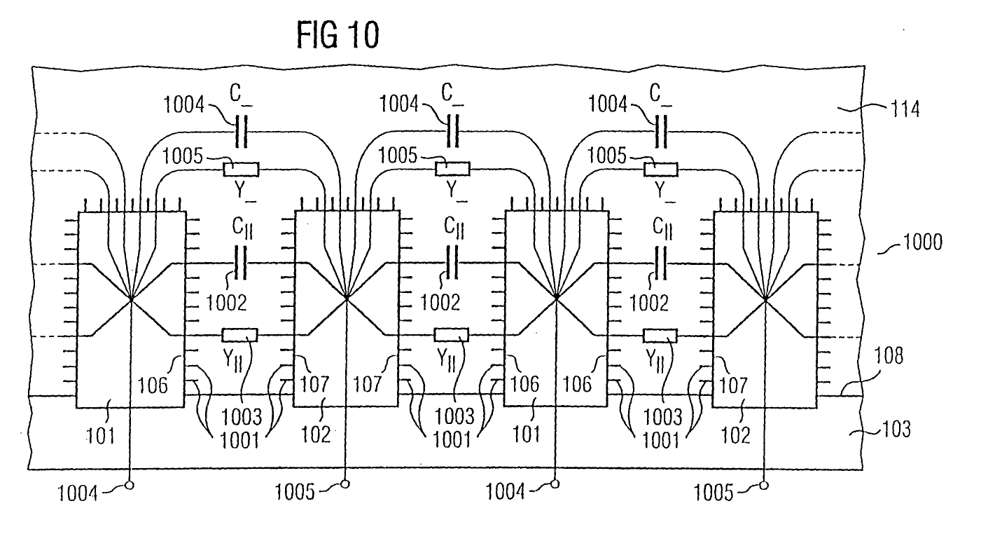

FIG. 10 shows a cross section through a biosensor with two electrodes which are arranged as an interdigitated electrode arrangement;

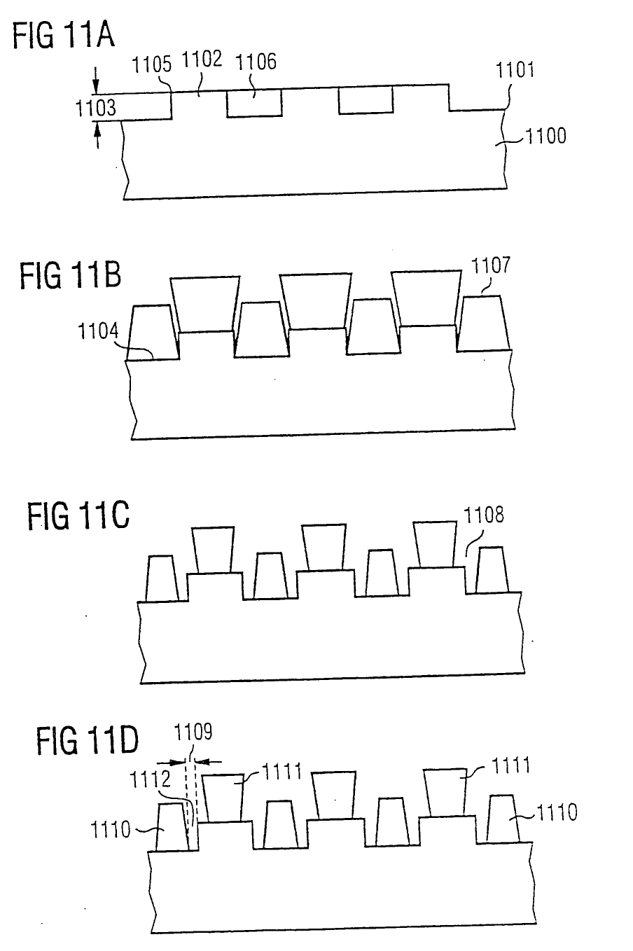

FIGS. 11a to 11d show cross-sectional views through an interdigitated electrode in four method states in a method for producing a biosensor in accordance with an exemplary embodiment of the invention;

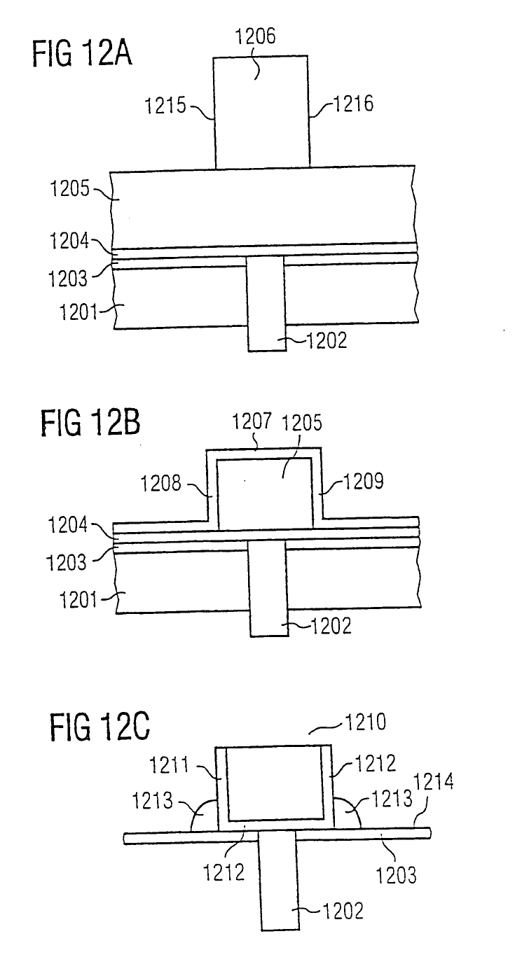

FIGS. 12a to 12c show cross-sectional views through a biosensor during individual method stages of the method for producing an electrode of the biosensor in accordance with a further exemplary embodiment of the invention;

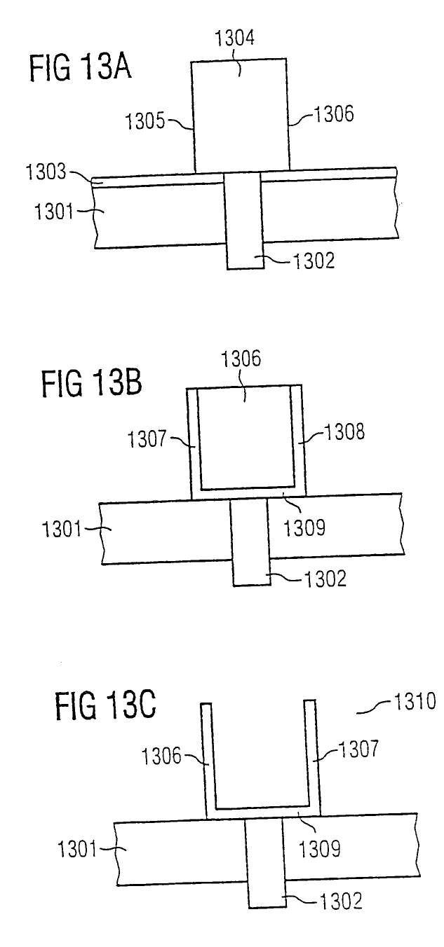

FIGS. 13a to 13c show cross-sectional views through a biosensor during individual method stages of the method for producing an electrode of the biosensor in accordance with a further exemplary embodiment of the invention;

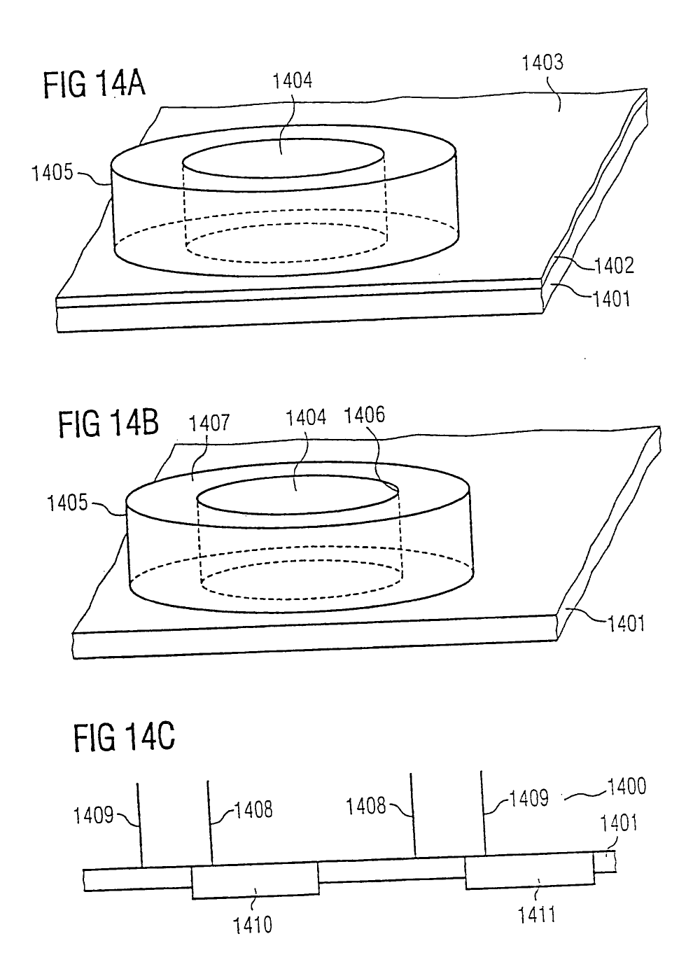

FIGS. 14a to 14c respectively show a cross section through a biosensor at various times during the production method in accordance with a further exemplary embodiment of the invention;

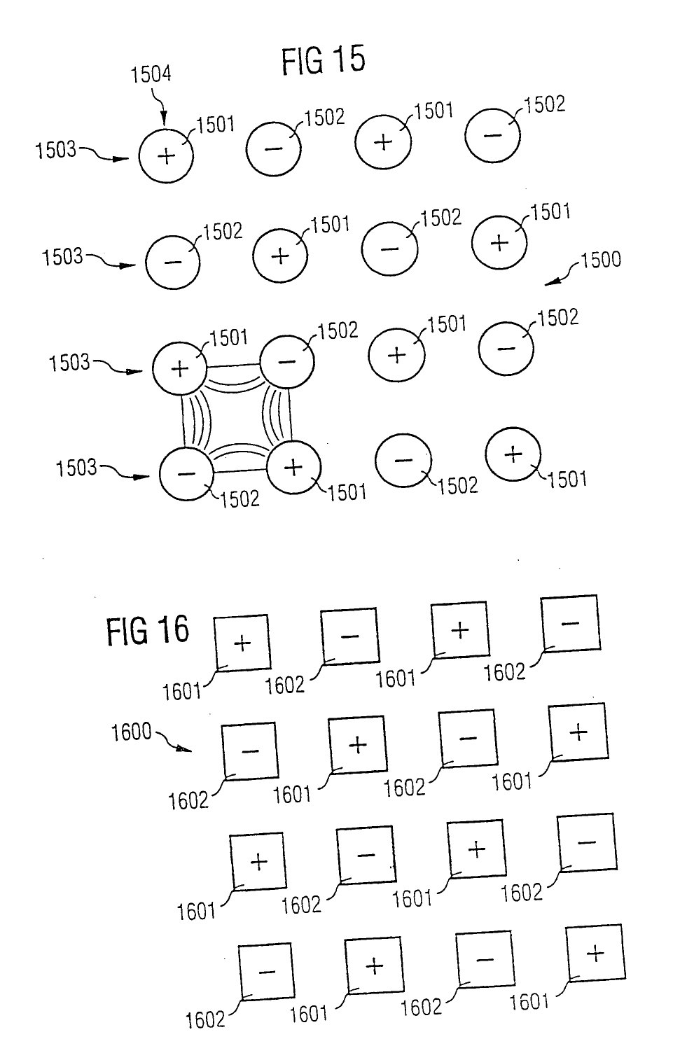

FIG. 15 shows a plan view of a biosensor array according to an exemplary embodiment of the invention with cylindrical electrodes;

FIG. 16 shows a plan view of a biosensor array in accordance with an exemplary embodiment of the invention with cuboidal electrodes;

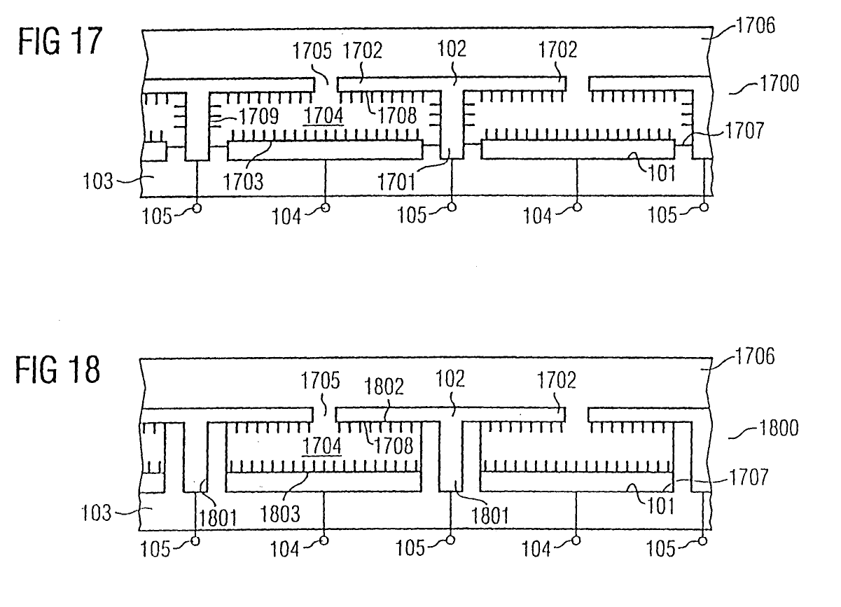

FIG. 17 shows a cross-sectional view through a biosensor in accordance with a further exemplary embodiment of the invention;

FIG. 18 shows a cross-sectional view through a biosensor in accordance with a further exemplary embodiment of the invention; and

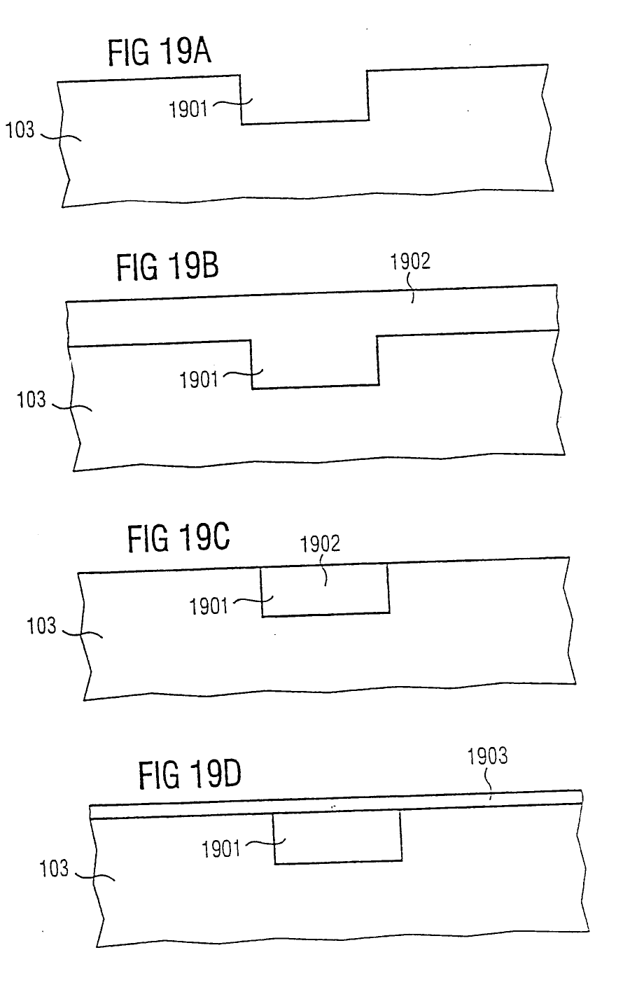

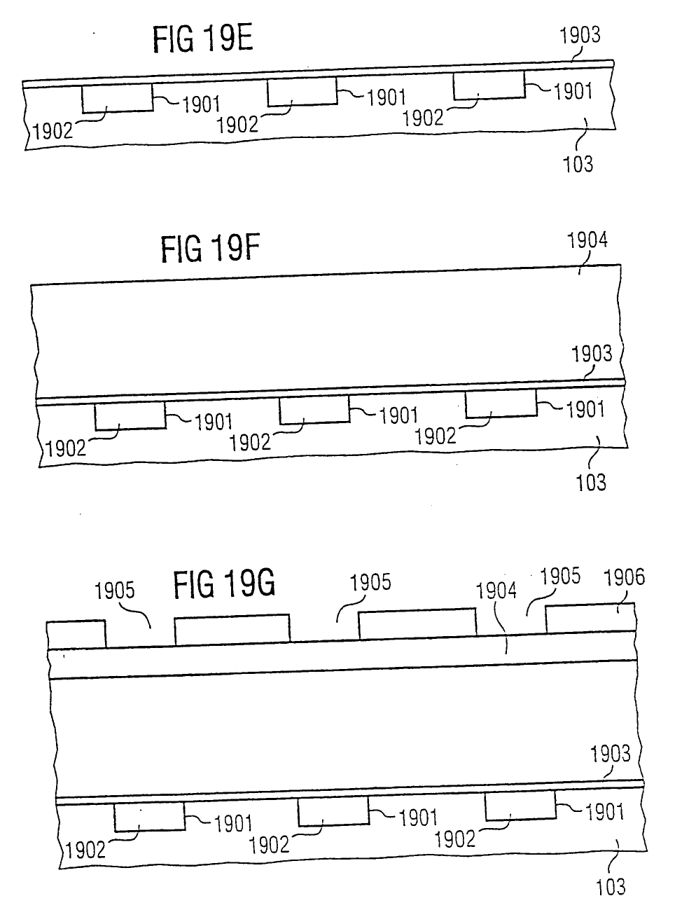

FIGS. 19a to 19g show cross-sectional views through a biosensor during individual method stages of a production method according to a further exemplary embodiment of the invention;

FIG. 1 shows a planar electrode arrangement on a biosensor chip 100 with a first electrode 101 and a second electrode 102, a holding region for holding DNA probe molecules being provided on the surface 103 of the first electrode 101, as is known from [1].

The first electrode 101 and the second electrode 102 are made from gold.

The first electrode 101 is assigned a first electrical potential V1 by means of a first voltage source 104.

The second electrode 102 is assigned a second electrical potential V2 by means of a second voltage source 105.

The first electrical potential V1 and the second electrical potential V2 are selected in such a manner that, in accordance with the method which has been explained in connection with the prior art, a reduction/oxidation operation is established when the electrodes 101, 102 are brought into contact firstly with a solution which is to be analyzed (not shown), then with a rinsing solution and finally with a solution with a substance which includes molecules which are cleaved by means of an enzyme which marks the hybridized DNA strands which are immobilized on the first electrode 101.

In accordance with this exemplary embodiment, by way of example the following can be used as enzyme:

a-galactosidase,

b-galactosidase,

b-glucosidase,

a-mannosidase,

alkaline phosphatase,

acidic phosphatase,

oligosaccharide dehydrogenase,

glucose dehydrogenase,

laccase,

tyrosinase,

or enzymes of related types.

It should be noted that low-molecular weight enzymes are able to ensure the highest conversion efficiency and therefore also the highest sensitivity.

Therefore, the further solution contains molecules which can be cleaved by the enzyme into a first part-molecule having a negative electric charge and into a second part-molecule having a positive electric charge.

The cleavable molecule used may, above all, be, for example:

p-aminophenyl hexopyranosides,

p-aminophenyl phosphates,

p-nitrophenyl hexopyranosides,

p-nitrophenyl phosphates, or

suitable derivatives of

a) diamines,

b) catecholamines,

c) Fe(CN)⁴⁻₆,

d) ferrocene,

e) dicarboxylic acid,

f) ferrocenelysine,

g) osmium bipyridyl-NH, or

h) PEG-ferrocene².

The resulting circuit current, indicated by directional arrows 106, 107 is recorded and converted into a first output voltage V_{OUT1} by means of a current-voltage converter 108, which is coupled to the second electrode.

The current-voltage converter 108 has a first operational amplifier 109, the non-inverting input 110 of which is coupled to the second voltage source 105 and the inverting input 111 of which is coupled to the second electrode 102.

The output 112 of the first operational amplifier 109 is fed back, via a first electrical resistance R1 113, to the inverting input 111 of the first operational amplifier 109.

Furthermore, the output 112 of the first operational amplifier 109 is coupled to a differentiator circuit 114, which is likewise integrated in the biosensor chip 100.

The differentiator circuit 114 has a capacitor C 115, a second operational amplifier 116 and a second electrical resistance R2 117.

The output of the first operational amplifier 112 is coupled to a first connection 118 of the capacitor C 115.

A second connection 119 of the capacitor C 115 is coupled to the inverting input 120 of the second operational amplifier 116.

The non-inverting input 121 of the second operational amplifier 116 is coupled to ground potential.

The output 122 of the second operational amplifier 116 is coupled via the second electrical resistance R2 117 to the inverting input 120 of the second operational amplifier 116.

Furthermore, the output 122 of the second operational amplifier 116 is coupled to an external electrical connection 123, at which a second output voltage V_{OUT2} of the biosensor chip 100 is made available.

This on-chip solution keeps the influences of noise signals to a low level, this occurring in particular on account of the determination of the increase m

$$m = \frac{ⅆ I}{ⅆ t} \qquad ( 2 )$$

from the sensor signal Iₛₑₙₛₒᵣ recorded by the second electrode 102, where

Iₛₑₙₛₒᵣ=I_{offset}+m·t (5)

in the immediate vicinity of the second electrode 102.

The first output voltage V_{OUT1} is present at the output 112 of the first operational amplifier 109 and results from the following rule:

V_{OUT1}=(I_{offset}+m·t)·R1+V2 (6).

Furthermore, the current-voltage converter 108 in the present circuit ensures that the second electrical potential V2 is present at the second electrode 102.

The effect of the downstream differentiator circuit 114 is that, on account of the first output voltage V_{OUT1}, an output signal, i.e. the second output voltage V_{OUT2}, is formed, this second output voltage being proportional to the increase m determined, in accordance with the following rule:

V_{OUT2}=m·C·R1·R2. (7).

Therefore, to determine the increase m, it is necessary to know the values for the capacitance C 115, the first electrical resistance R1 113 and the second electrical resistance R2 117.

According to the first exemplary embodiment, the levels of the resistances R1 113, R2 117 and of the capacitance C 115 can be measured directly on the biosensor chip 100.

In this way, the calibration of the biosensor chip 100 and, on the basis of this, the recording of the measured values can take place.

According to one configuration of the invention, frequency-band limitation is connected upstream of the differentiator circuit 114, for example by means of a low-pass.

However, in order to be able to counteract possible fluctuations in the levels for different biosensor chips and different wafers resulting from changing production conditions, according to a second exemplary embodiment a reference circuit 600 (cf. FIG. 6a) is provided.

The reference circuit 600 has the same structure as the differentiator circuit 114, i.e. a capacitor C 601, an operational amplifier 602 and an electrical resistance R 603.

A first connection 604 of the capacitor is coupled to the inverting input 605 of the operational amplifier 602.

The non-inverting input 606 of the operational amplifier 602 is coupled to ground potential.

The output 607 of the operational amplifier 606 is fed back via the electrical resistance 603 to the inverting input 605 of the operational amplifier 602.

In addition, the reference circuit 600 optionally, i.e. if a low-pass filter is connected upstream of the differentiator circuit 114, has a low-pass filter 608 for filtering out high-frequency signals, in particular the noise signals.

The first connection 609 of the low-pass filter 608 is coupled to the input 610 of the reference circuit 600, and the second connection 611 of the low-pass filter 608 is connected to the second connection 612 of the capacitor C 601.

The output 607 of the operational amplifier 602 is coupled to the output 613 of the reference circuit 600.

FIG. 6b shows a Bode diagram 620 of the low-pass filtering of an input signal V_{IN} which is brought about by means of the low-pass 608 in order to determine an output signal V_{OUT} as a function of a cutoff frequency f_{G} of the low-pass filter 608.

The profile of the output voltage V_{OUT} as a function of a frequency f is diagrammatically depicted in the Bode diagram 620 as a curve 621.

FIG. 7 shows the biosensor chip 700 in accordance with an alternative embodiment having the reference circuit 600.

As illustrated in FIG. 7, the reference circuit 600 is arranged in the immediate vicinity, i.e. at a distance of approximately a few micrometers on the biosensor chip 700, with respect to the electrode arrangement, and in particular with respect to the differentiator circuit 114 and the current-voltage converter 108.

A current source 701, which is used to supply a reference current I_{ref} 702 to the reference circuit 600, is coupled to an input 610 of the reference circuit 600.

The reference current I_{ref} 702 results in accordance with the following rule:

I_{ref}=m_{ref}·t (8).

As explained below, on account of this alternative embodiment, it is no longer necessary for the values for the differentiator circuit 114, i.e. of the capacitance 115 and of the second electrical resistance R2 117 and the value of the first electrical resistance R1 113 of the current-voltage converter 108 to be measured.

It should be noted that the reference circuit 600 and the differentiator circuit 114 have basically identical layouts.

Therefore, a reference output voltage V_{OUT2, ref} results at the output 613 of the reference circuit 600 in accordance with the following rule:

V_{OUT2, ref}=m_{ref}·C·R1·R2 (9).

The output of the biochip sensor from FIG. 1123 is coupled to a first connection 703 of an evaluation unit 704.

Furthermore, the output 613 of the reference circuit 600 is coupled to a second input 705 of the evaluation unit 704.

The increase m is determined in the evaluation unit 704 in accordance with the following rule:

$$m = {m}_{ref} \cdot \frac{{V}_{OUT2}}{{V}_{OUT2 , ref}} . \qquad ( 10 )$$

The increase m which is to be determined is made available as output signal from the evaluation unit 704 at the output 706 thereof.

The number of hybridized DNA strands marked with the enzyme which have hybridized with the DNA probe molecules on the first electrode 101 can now be determined from the increase m in the known way.

The measuring instrument used to determine the output signal of the evaluation unit 704, which is present as output voltage at the output 706 of the evaluation unit 704, can then be recorded by means of a simple voltmeter.

It should be noted that as an alternative the evaluation unit 704 may likewise be integrated in the biosensor chip 700.

Furthermore, it should be noted that the invention is not restricted to a biosensor chip for recording DNA molecules, but rather, by suitably changing the first electrode 101, i.e. by immobilizing ligands at the first electrode 101, it is also possible to record other macromolecular biopolymers which are marked with the enzyme, with the result that it is likewise possible to achieve a reduction/oxidation recycling operation, as has been explained above.

Furthermore, it should be noted that the invention is not restricted to a planar electrode arrangement.

The electrodes may be arranged in the form of an interdigitated electrode arrangement, as described in [4].

Furthermore, alternative electrode configurations, as explained below, may be arranged on the biosensor chip 100, 700.

FIG. 9 shows a biosensor chip 900 with a further electrode configuration.

The biosensor chip 900 has a first electrode 901 and a second electrode 902, which are arranged on an insulator layer 903 in such a way that the first electrode 901 and the second electrode 902 are electrically insulated from one another.

The first electrode 901 is coupled to a first electrical terminal 904, and the second electrode 902 is coupled to a second electrical terminal 905.

The electrodes 901, 902 have a cuboid structure, with a first electrode face 906 of the first electrode 901 and a first electrode face 907 of the second electrode 902 facing one another while being aligned essentially parallel.

This is achieved, according to this exemplary embodiment, by the fact that the electrodes 901, 902 have side walls 906, 907 which are essentially perpendicular with respect to the surface 108 of the insulator layer 903, and which respectively form the first electrode face 906 of the first electrode 901 and the first electrode face 907 of the second electrode 902.

If an electric field is applied between the first electrode 901 and the second electrode 902, then owing to the electrode faces 906, 907 which are aligned essentially parallel with one another, a field line profile is produced with field lines 909 which are essentially uncurved between the faces 906, 907.

Curved field lines 910 occur only between a second electrode face 911 of the first electrode 901 and a second electrode face 912 of the second electrode 902, which respectively form the upper surfaces for the electrodes 901, 902, as well as in an edge region 913 between the electrodes 901, 902.

The first electrode faces 906, 907 of the electrodes 901, 902 are formed as holding regions for holding probe molecules, which can bind macromolecular biopolymers that are detected by means of the biosensor 900.

The electrodes 901, 902 are made of gold according to this exemplary embodiment.

Covalent bonds are produced between the electrodes and the probe molecules, the sulfur for forming a gold-sulfur coupling being present in the form of a sulfide or a thiol.

For the case in which DNA probe molecules are used as the probe molecules, such sulfur functionalities are part of a modified nucleotide which is incorporated by means of phosphoramidite chemistry during an automated DNA synthesis method at the 3′ end or at the 5′ end of the DNA strand to be immobilized. The DNA probe molecule is therefore immobilized at its 3′ end or at its 5′ end.

For the case in which ligands are used as the probe molecules, the sulfur functionalities are formed by one end of an alkyl linker or of an alkylene linker, the other end of which has a chemical functionality suitable for the covalent bonding of the ligand, for example a hydroxyl radical, an acetoxy radical or a succinimidyl ester radical.

The electrodes, i.e. in particular the holding regions, are covered during measurement use with an electrolyte 914, in general with a solution to be analyzed.

If the solution 914 to be analyzed contains the macromolecular biopolymers to be recorded, for example DNA strands to be recorded which have a predetermined sequence and which can hybridize with the immobilized DNA probe molecules on the electrodes, then the DNA strands hybridize with the DNA probe molecules.

If the solution 914 to be analyzed does not contain any DNA strands with the sequence complementary to the sequence of the DNA probe molecules, then no DNA strands from the solution 914 to be analyzed can hybridize with the DNA probe molecules on the electrodes 901, 902.

As has been explained above, a redox recycling operation is started between the electrodes 901, 902, and in this way the number of marked hybridized DNA strands, generally of the marked, bound macromolecular biopolymers is determined.

FIG. 10 shows a biosensor 1000 with a further electrode configuration according to a further exemplary embodiment of the invention.

In the biosensor 1000, in the same way as in the biosensor 900 according to the exemplary embodiment shown in FIG. 9, two electrodes 901, 902 are provided which are applied on the insulator layer 903.

In contrast to the biosensor 900 with only two cuboid electrodes, the two electrodes according to the biosensor 900 represented in FIG. 10 are arranged as a plurality of respectively alternately arranged, parallel-connected electrodes in the form of the known interdigitated electrode arrangement.

For further illustration, FIG. 10 also shows a schematic electrical equivalent circuit diagram, which is indicated in the representation of the biosensor 1000.

Since essentially uncurved field lines occur with respect to the surface 908 of the insulator layer 903 between the electrode faces 906, 907 of the electrodes 901, 902, which face one another while being essentially parallel, as was represented in FIG. 9, the component of the first capacitance 1002 and of the first admittance 1003 produced by the uncurved field lines predominates compared with the second capacitance 1004 and the second admittance 1005, which are produced by the curved field lines 910.

This significantly greater component of the first capacitance 1002 and of the first admittance 1003 in relation to the total admittance, which is obtained from the sum of the first capacitance 1002 and the second capacitance 1004 as well as the first admittance 1003 and the second admittance 1005, has the effect of significantly increasing the sensitivity of the biosensor 1000 when the state of the biosensor 1000 changes, i.e. when DNA strands in the solution 914 to be analyzed hybridize with DNA probe molecules 1001 immobilized on the holding regions on the electrode faces 906, 907.

Clearly, with the same lateral dimensions of the electrodes 901, 902 and the same dimensions of the previously introduced active region, i.e. with the same area of the holding regions on the electrode faces, a substantially greater component of field lines of an applied electric field between the electrodes 901, 902 is therefore contained in the volume in which the hybridization takes place when DNA strands to be recorded are contained in the solution 914 to be analyzed, than in the case of a planar electrode arrangement.

In other words, this means that the capacitance of the arrangement according to the invention per unit chip area is significantly greater than the capacitance per unit chip area in the case of a planar electrode arrangement.

A few alternative possibilities for producing a cuboid sensor electrode with essentially vertical side walls will be explained below.

First method for producing metal electrodes with essentially vertical side walls, which can immobilize probe molecules

FIG. 11a shows a silicon substrate 1100, as is produced for known CMOS processes.

On the silicon substrate 1100, which already contains integrated circuits and/or electrical terminals for the electrodes to be formed, an insulator layer 1101 which is also used as a passivation layer is applied with a sufficient thickness, with a thickness of 500 nm according to the exemplary embodiment, by means of a CVD method. The insulator layer 1101 may be made of silicon oxide SiO₂ or silicon nitride Si₃N₄.

The interdigitated arrangement of the biosensor 1000 according to the exemplary embodiment described above is defined by means of photolithography on the insulator layer 1101.

By means of a dry etching method, e.g. reactive ion etching (RIE), steps 1102 are subsequently produced, i.e. etched, in the insulator layer 1101 with a minimum height 1103 of approximately 100 nm according to the exemplary embodiment.

The height 1103 of the steps 1102 must be large enough for a subsequent self-aligning process to form the metal electrode.

It should be pointed out that an evaporation coating method or a sputtering method may alternatively also be used for applying the insulator layer 1101.

During the structuring of the steps 1102, care should be taken that the flanks of the steps 1102 are steep enough so that they form sufficiently sharp edges 1105. An angle 1106 of the step flanks, measured with respect to the surface of the insulator layer 1101, should be at least 50 degrees according to the exemplary embodiment. In a further step, an auxiliary layer 1104 (cf. FIG. 11b) made of titanium with a thickness of approximately 10 nm is applied to the stepped insulator layer 1101.

The auxiliary layer 1104 may comprise tungsten and/or nickel-chromium and/or molybdenum.

It is necessary to guarantee that the metal layer applied in a further step, according to the exemplary embodiment a metal layer 1107 made of gold, grows porously at the edges 1105 of the steps 1102 so that, in a further method step, it is possible to respectively etch a gap 1108 at the step junctions, into the gold layer 1107 which is applied surface-wide.

The gold layer 1107 for the biosensor 1000 is applied in a further method step.

According to the exemplary embodiment, the gold layer has a thickness of from approximately 500 nm to approximately 2000 nm.

In terms of the thickness of the gold layer 1107, it is merely necessary to guarantee that the thickness of the gold layer 1107 is sufficient for the gold layer 1107 to grow porously in columns.

In a further step, openings 1108 are etched into the gold layer 1107 so that gaps are formed.

For wet etching of the openings, an etchant solution made up of 7.5 g of Super Strip 100™ (trademark of Lea Ronal GmbH, Germany) and 20 g of KCN in 1000 ml of water H₂O is used.

Owing to the columnar growth of the gold, in general of the metal, during the evaporation coating onto the adhesion layer 1104, anisotropic etching attack is achieved so that the surface erosion of the gold takes place approximately in the ratio 1:3.

The gaps 1108 are formed as a function of the duration of the etching process by the etching of the gold layer 1107.

This means that the duration of the etching process dictates the basic width, i.e. the distance 1109 between the gold electrodes 1110, 1111 which are being formed.

After the metal electrodes have a sufficient width and the distance 1109 between the gold electrodes 1110, 1111 which are being formed is achieved, the wet etching is ended.

It should be noted that, because of the porous evaporation coating, etching in a direction parallel to the surface of the insulator layer 1101 takes place substantially faster than in a direction perpendicular to the surface of the insulator layer 1101.

It should be pointed out that alternatively to a gold layer, it is possible to use other noble metals, for example platinum, titanium or silver, since these materials can likewise have holding regions or can be coated with a suitable material for holding immobilized DNA probe molecules, or in general for holding probe molecules, and they exhibit columnar growth during evaporation coating.

For the case in which the adhesion layer 1104 needs to be removed in the opened columns 1112 between the metal electrodes 1110, 1111, this is likewise carried out in a self-aligning fashion by using the gold electrodes 1110, 1111 as an etching mask.

Compared with the known interdigitated electrodes, the structure according to this exemplary embodiment has the advantage, in particular, that owing to the self-aligning opening of the gold layer 1107 over the edges 1105, the distance between the electrodes 1110, 1111 is not tied to a minimum resolution of the production process, i.e. the distance 1109 between the electrodes 1110, 1111 can be kept very narrow.

According to this method, the biosensor 1000 according to the exemplary embodiment represented in FIG. 10 with the corresponding metal electrodes is therefore obtained.

Second method for producing metal electrodes with essentially vertical side walls, which can immobilize probe molecules

The production method represented in FIG. 12a to FIG. 12c starts with a substrate 1201, for example a silicon substrate wafer (cf. FIG. 12a), on which metallization 1202 is already provided as an electrical terminal, an etch stop layer 1203 of silicon nitride Si₃N₄ already having been applied on the substrate 1201.

A metal layer 1204, according to the exemplary embodiment a gold layer 1204, is applied on the substrate by means of an evaporation coating method.

Alternatively, a sputtering method or a CVD method may also be used to apply the gold layer 1204 to the etch stop layer 1203.

In general, the metal layer 1204 comprises the metal on which the electrode to be formed is intended to be formed.

An electrically insulating auxiliary layer 1205 of silicon oxide SiO₂ is applied on the gold layer 1204 by means of a CVD method (alternatively by means of an evaporation coating method or a sputtering method).

By using photolithographic technology, a resist structure, for example a cuboid structure, is formed from a resist layer 1206, which corresponds to the shape of the electrode to be formed.

If a biosensor array, described below, with a plurality of electrodes is to be produced, a resist structure whose shape corresponds to the electrodes to be formed, which form the biosensor array, is produced by means of photolithography.

Put another way, this means that the lateral dimensions of the resist structure which is formed correspond to the dimensions of the sensor electrode to be produced.

After application of the resist layer 1206 and the corresponding illumination, which defines the corresponding resist structures, the resist structure is removed in the “undeveloped”, i.e. unilluminated regions, for example by means of ashing or wet chemically.

The auxiliary layer 1205 is also removed by means of a wet etching method or a dry etching method in the regions not protected by the photoresist layer 1206.

In a further step, after removal of the resist layer 1206, a further metal layer 1207 is applied conformally as an electrode layer over the remaining auxiliary layer 1205, in such a way that the side faces 1208, 1209 of the residual auxiliary layer 1205 are covered with the electrode material, according to the exemplary embodiment with gold (cf. FIG. 12b).

The application may be carried out by means of a CVD method or a sputtering method or by using an ion metal plasma method.

In a last step (cf. FIG. 12c), spacer etching is carried out, during which the desired structure of the electrode 1210 is formed by deliberate over-etching of the metal layers 1204, 1207.

The electrode 1210 therefore has the spacers 1211, 1212, which have not been etched away in the etching stage of etching the metal layers 1204, 1207, as well as the part of the first metal layer 1204, arranged immediately below the residual auxiliary layer 1205, which has not been etched away by means of the etching method.

The electrode 1210 is electrically coupled to the electrical terminal, i.e. the metallization 1202.

The auxiliary layer 1205 of silicon oxide may if necessary be removed by further etching, for example in the plasma or wet chemically, by means of a method in which selectivity with respect to the etch stop layer 1203 is provided.

This is guaranteed, for example, if the auxiliary layer 1205 consists of silicon oxide and the etch stop layer 1203 comprises silicon nitride.

The steepness of the walls of the electrode in the biosensor chip 900, 1000, represented by the angle 1213 between the spacers 1211, 1212 and the surface 1214 of the etch stop layer 1203, is therefore determined by the steepness of the flanks of the residual auxiliary layer 1205, i.e. in particular the steepness of the resist flanks 1215, 1216 of the structured resist layer 1206.

Third method for producing metal electrodes with essentially vertical side walls, which can immobilize probe molecules

FIG. 13a to FIG. 13c represent a further possibility for producing an electrode within essentially vertical walls.

This also, as represented in the second example of producing an electrode, starts with a substrate 1301 on which a metallization 1302 is already provided for the electrical terminal of the biosensor electrode to be formed.

A metal layer 1303 is evaporation-coated as an electrode layer on the silicon substrate 1301, the metal layer 1303 comprising the material to be used for the electrode, according to this exemplary embodiment gold.

Alternatively to evaporation coating of the metal layer 1303, the metal layer 1303 may also be applied on the substrate 1301 by means of a sputtering method or by means of a CVD method.

A photoresist layer 1304 is applied on the metal layer 1303 and is structured by means of photolithographic technology so as to produce a resist structure which, after development and removal of the developed regions, corresponds to the lateral dimensions of the electrode to be formed, or in general of the biosensor array to be formed.

The thickness of the photoresist layer 1304 corresponds essentially to the height of the electrodes to be produced.

During structuring in a plasma with process gases which cannot lead to any reaction of the electrode material, in particular in an inert gas plasma, for example with argon as the process gas, the erosion of the material according to this exemplary embodiment is carried out by means of physical sputter erosion.

In this case, the electrode material is sputtered from the metal layer 1303 in a redeposition process onto the essentially vertical side walls 1305, 1306 of the structured resist elements that are not removed after ashing the developed resist structure, where it is no longer exposed to any sputter attack.

Redeposition of electrode material onto the resist structure protects the resist structure from further erosion.

Because of the sputtering, side layers 1307, 1308 of the electrode material, according to the exemplary embodiment of gold, are formed at the side walls 1305, 1306 of the resist structure.

The side layers 1307, 1308 are electrically coupled to an unremoved part 1309 of the metal layer 1303, which lies immediately below the residual resist structure 1306, and furthermore to the metallization 1303 (cf. FIG. 13b).

In a last step (cf. FIG. 13c), the resist structure 1306, i.e. the photoresist which is found in the volume formed by the side walls 1307, 1308 as well as the remaining metal layer 1309, is removed by means of ashing or wet chemically.

The result is the electrode structure 1310 represented in FIG. 13c, which is formed with the side walls 1307, 1308 as well as the unremoved part 1309, which forms the bottom of the electrode structure and is electrically coupled to the metallization 1303.

As in the production method presented above, the steepness of the side walls 1307, 1308 of the electrode that is formed in this method is determined by the steepness of the resist flanks 1305, 1306.

FIG. 14a to FIG. 14c represent a further exemplary embodiment of the invention with cylindrical electrodes protruding perpendicularly from the substrate.

In order to produce the biosensor 1400 with cylindrical electrodes, which are arranged essentially perpendicularly on a substrate 1401 of silicon oxide, a metal layer 1402 is applied by means of an evaporation coating method as an electrode layer of the desired electrode material, according to the exemplary embodiment of gold.

A photoresist layer is applied on the metal layer 1402, and the photoresist layer is illuminated by means of a mask so that the cylindrical structure 1403 represented in FIG. 14a is obtained on the metal layer 1402 after the unilluminated regions have been removed.

The cylindrical structure 1403 has a photoresist torus 1404 as well as a cylindrical photoresist ring 1405, which is arranged concentrically around the photoresist torus 1404.

The photoresist is removed between the photoresist torus 1404 and the photoresist ring 1405, for example by means of ashing or wet chemically.

Through the use of a sputtering method, as in conjunction with the method described above for producing an electrode, a metal layer 1406 is applied around the photoresist torus 1404 by means of a redeposition process.

In a similar way, an inner metal layer 1407 is formed around the photoresist ring 1405 (cf. FIG. 14b).

In a further step, the structured photoresist material is removed by means of ashing or wet chemically, so that two cylindrical electrodes 1408, 1409 are formed.

The substrate 1401 is removed in a last step, for example by means of a plasma etching process that is selective with respect to the electrode material, to the extent that the metallizations in the substrate are exposed and electrically couple to the cylindrical electrodes.

The inner cylindrical electrode 1408 is therefore electrically coupled to a first electrical terminal 1410, and the outer cylindrical electrode 1409 is electrically coupled to a second electrical terminal 1411.

The residual metal layer 1402, which has not yet been removed by the sputtering between the cylindrical electrodes 1408, 1409, is removed in a last step by means of a sputter-etching process. The metal layer 1402 is likewise removed in this way.

It should be mentioned in this context that, according to this exemplary embodiment as well, the metallizations for the electrical terminals 1410, 1411 are already provided in the substrate 1401 at the start of the method.

FIG. 15 shows a plan view of a biosensor array 1500, in which cylindrical electrodes 1501, 1502 are contained.

Each first electrode 1501 has a positive electrical potential.

Each second electrode 1502 of the biosensor array 1500 has an electrical potential that is negative in relation to the respectively neighboring first electrode 1501.

The electrodes 1501, 1502 are arranged in rows 1503 and columns 1504.

The first electrodes 1501 and the second electrodes 1502 are respectively arranged alternately in each row 1503 and each column 1504, i.e. a second electrode 1502 is respectively arranged in a row 1503 or a column 1504 immediately next to a first electrode 1501, and a first electrode 1501 is respectively arranged in a row 1503 or a column 1504 next to a second electrode 1502.

This ensures that an electric field with essentially uncurved field lines in the height direction of the cylinder electrodes 1501, 1502 can be produced between the individual electrodes.

As described above, a large number of DNA probe molecules are respectively immobilized on the electrodes.

If a solution to be analyzed (not shown) is then applied to the biosensor array 1500, then the DNA strands hybridize with DNA probe molecules complementary thereto which are immobilized on the electrodes.

In this way, by means of the redox recycling operation described above, the existence or nonexistence of DNA strands of a predetermined sequence in a solution to be analyzed can in turn be detected by means of the biosensor array 1500.

FIG. 16 shows a further exemplary embodiment of a biosensor array 1600 with a plurality of cuboid electrodes 1601, 1602.

The arrangement of the cuboid electrodes 1601, 1602 is in accordance with the arrangement of the cylindrical electrodes 1501, 1502 as presented in FIG. 15 and explained above.

FIG. 17 shows an electrode arrangement of a biosensor chip 1700 according to a further exemplary embodiment of the invention.

The first electrode 901 is applied on the insulator layer 903 and is electrically coupled to the first electrical terminal 904.

The second electrode 902 is likewise applied on the insulator layer 903 and is electrically coupled to the second electrical terminal 905.

As shown in FIG. 17, the second electrode according to this exemplary embodiment has a different shape compared with the second electrode described previously.

The first electrode, as can be seen from FIG. 17, is a planar electrode and the second electrode is configured with a T-shape.

Each T-shaped second electrode has a first branch 1701, which is arranged essentially perpendicular to the surface 1707 of the insulator layer 903.

Furthermore, the second electrode 902 has second branches 1702 which are arranged perpendicular to the first branch 1701 and are arranged at least partially over the surface 1703 of the respective first electrode 901.

As can be seen in FIG. 17, several first electrodes 901 and several second electrodes 902 are connected in parallel, so that because of the T-shaped structure of the second electrode 902, a cavity 1704 is created which is formed by two second electrodes 902 arranged next to one another, one first electrode 901 and the insulator layer 903.

The individual first and second electrodes 901, 902 are electrically insulated from one another by means of the insulator layer 903.

An opening 1705 is provided between the individual second branches 1702 of the second electrode 902 for each cavity 1704, which opening 1705 is large enough so that when an electrolyte 1706 is being applied to the biosensor 1700, the electrolyte and DNA strands possibly contained in the solution 1706 to be analyzed, for example an electrolyte, can pass through the opening 1705 into the cavity 1704.

DNA probe molecules 1709, which can hybridize with the corresponding DNA strands of a predetermined sequence that are to be recorded, are immobilized on holding regions on the first and second electrodes.

As can be seen in FIG. 17, because of the mutually facing surfaces, aligned essentially parallel with one another, of the second electrode 1708 and of the first electrode 1703, on which the holding regions for holding the DNA probe molecules 1709 are provided, essentially uncurved field lines are formed when an electric field is applied between the first electrode 901 and the second electrode 902.

FIG. 18 shows a biosensor 1800 according to a further exemplary embodiment of the invention.

The biosensor 1800 according to the further exemplary embodiment corresponds essentially to the biosensor 1700 explained above and shown in FIG. 17, with the difference that no holding regions with immobilized DNA probe molecules 1709 are provided on side walls of the first branch 1701 of the second electrode 902, but rather the surface 1801 of the first branch 1701 of the second electrode 902 is covered with insulator material of the insulator layer 903 or a further insulating layer.

According to the exemplary embodiment shown in FIG. 18, holding regions on the first electrode and on the second electrode 901, 902 are consequently only on directly facing surfaces of the electrodes, i.e. on the surface 1802 of the second branch of the second electrode 902 and on the surface 1803 of the first electrode 901.

FIG. 19a to FIG. 19g represent individual method steps for producing the first electrode 901 and the second electrode 902 in the biosensors 1700, 1800.

In the insulator layer 903 as a substrate, according to the exemplary embodiment made of silicon oxide, a structure whose shape corresponds to the first electrode 901 to be formed is etched into the insulator layer 903 by using a mask layer, for example made of photoresist.

After removal of the mask layer by ashing or by a wet chemical method, a layer of the desired electrode material is applied surface-wide on the insulator layer 903, in such a way that the previously etched structure 1901 (cf. FIG. 19a) is at least completely filled; the structure 1901 may even be overfilled (cf. FIG. 19b).

In a further step, the electrode material 1902, preferably gold, located outside the prefabricated structure 1901 is removed by means of a chemical-mechanical polishing method (cf. FIG. 19c).

After the completion of the chemical-mechanical polishing method, the first electrode 901 is therefore embedded flush in the insulator layer 903.

Electrode material 1902 outside, i.e. between the further second electrodes 902 or between the first electrodes 901, is removed without leaving any residue.

A cover layer 1903, for example made of silicon nitride, may furthermore be applied to the first electrode 901 by means of a suitable coating method, for example a CVD method, a sputtering method or an evaporation coating method (cf. FIG. 19d).

FIG. 19e shows several first electrodes 1901 made of gold, which are embedded next to one another in the insulator layer 903, and the cover layer 1903 located on top.

In a further step (cf. FIG. 19f), a second electrode layer 1904 is applied on the cover layer 1903.

After masking has been completed, taking account of the desired opening between the second electrodes, which is to be formed from the second electrode layer 1904, the desired openings 1905 are formed, and the second electrode layer 1904 is etched by means of a dry etching process in a downstream plasma, in such a manner that the desired cavity 1704 is formed in accordance with the biosensors 1700, 1800 illustrated in FIG. 17 or FIG. 18 (cf. FIG. 19g).

It should be noted in this context that the cover layer 1903 is not absolutely indispensable, but it is advantageous in order to protect the first electrode 901 from superficial etching during the formation of the cavity 1704.

In an alternative embodiment, the T-shaped structure of the second electrode 902 may be formed as follows: after forming the first electrode 901 according to the method described above, a further insulator layer is formed by means of a CVD method or another suitable coating method on the first insulator layer or, if the cover layer 1903 exists, on the cover layer 1903. Subsequently, corresponding trenches are formed in the cover layer 1903, which are used to accommodate the first branch 1701 of the T-shaped structure of the second electrode 902. These trenches are filled with the electrode material gold and, according to the damascene method, the electrode material is removed which has been formed in the trenches and above the second insulator layer by means of chemical-mechanical polishing, down to a predetermined height which corresponds to the height of the second branch 1702 of the T-shaped second electrode 902.

The opening 1705 between the second electrodes 902 is formed by means of photolithography, and the insulator material is subsequently removed, at least partially, by means of a dry etching method in a downstream plasma from the volume which is intended to be formed as the cavity 1704.

It should furthermore be pointed out that the embodiments described above are not restricted to an electrode whose holding region is produced by means of gold. Alternatively, electrodes made from silicon monoxide or silicon dioxide may be used which are coated with materials in the holding regions. These materials-for example known alkoxysilane derivatives-may include amine, hydroxyl, epoxy, acetoxy, isocyanate or succinimidyl ester functionalities which are able to form a covalent bond with probe molecules which are to be immobilized, in this variant in particular ligands.

The following publications are cited in the present document:

[1] R. Hintsche et al., Microbiosensors Using Electrodes Made in Si-Technology, Frontiers in Biosensorics, Fundamental Aspects, edited by F. W. Scheller et al., Dirk Hauser Verlag, Basle, pp. 267-283, 1997.

[2] M. Paeschke et al, Voltammetric Multichannel Measurements Using Silicon Fabricated Microelectrode Arrays, Electroanalysis, Vol. 7, No. 1, pp. 1-8, 1996.

[3] R. Hintsche et al, Microbiosensors using electrodes made in Si-technology, Frontiers in Biosensorics, Fundamental Aspects, edited by F. W. Schaller et al, Birkhauser Verlag, Basle, Switzerland, 1997.

[4] P. van Gerwen, Nanoscaled Interdigitated Electrode Arrays for Biochemical Sensors, IEEE, International Conference on Solid-State Sensors and Actuators, Chicago, pp. 907-910, Jun. 16-19, 1997.

In the claims:

1. A biosensor chip, comprising:
a first electrode, which has a holding region for holding probe molecules which can bind macromolecular biopolymers;
a second electrode, the first electrode and the second electrode being designed in such a manner that a reduction/oxidation recycling operation can take place at these electrodes; and
an integrated electrical differentiator circuit, by means of which an electric current generated during the reduction/oxidation recycling operation can be recorded and can be differentiated over the course of time.

2. The biosensor chip as claimed in claim 1, further comprising:
a third electrode, the second electrode and the third electrode being designed in such a manner that the reduction/oxidation process takes place as part of the reduction/oxidation recycling operation at the second electrode and at the third electrode.

3. The biosensor chip as claimed in claim 2,
in which the first electrode has a first electrical potential;
in which the second electrode has a second electrical potential; and
in which the third electrode has a third electrical potential, the third electrical potential being selected in such a manner that during the reduction/oxidation recycling operation the reduction or oxidation takes place only at the second electrode and at the third electrode.

4. The biosensor chip as claimed in claim 3,
in which the third electrical potential is greater than the first electrical potential, and
in which the first electrical potential is greater than the second electrical potential.

5. The biosensor chip as claimed in claim 1 in which the holding region of the first electrode is coated with a material which can immobilize probe molecules.

6. The biosensor chip as claimed in claim 1 in which the holding region of the first electrode is designed to hold ligands, to which peptides or proteins can be bound.

7. The biosensor chip as claimed in claim 1 in which the holding region of the first electrode is designed to hold DNA probe molecules, to which DNA molecules can be bound.

8. The biosensor chip as claimed in claim 1 in which the holding region includes at least one of the following materials selected from the group consisting of hydroxyl radicals, epoxy radicals, amine radicals, acetoxy radicals, isocyanate radicals, succinimidyl ester radicals, thiol radicals, gold, silver, platinum, and titanium.

9. The biosensor chip as claimed in claim 1 in which the electrodes are arranged in an interdigitated electrode arrangement, the third electrode in each case being arranged between the first electrode and the second electrode.

10. The biosensor chip as claimed in claim 1 in which the first electrode and the second electrode and/or the third electrode are arranged in such a manner relative to one another that substantially uncurved field lines of an electric field, which is produced between the first electrode and the second electrode and/or the third electrode, can form between the first electrode and the second electrode and/or the third electrode.

11. The biosensor chip as claimed in claim 1 in which the differentiator circuit is electrically coupled to the second electrode.

12. The biosensor chip as claimed in claim 11, in which the differentiator circuit is electrically coupled to the second electrode via a current-voltage converter.

13. The biosensor chip as claimed in claim 1 having a reference circuit which has the same structure as the differentiator circuit and which can be used to generate an electrical reference signal.

14. The biosensor chip as claimed in claim 13, having an evaluation unit for evaluating the electrical signals generated by the differentiator circuit and by the reference circuit, it being possible to determine the increase in the curve of the electrical current generated during the reduction/oxidation recycling operation as a function of time using the evaluation unit.

15. The biosensor chip as claimed in claim 1 further comprising:
a multiplicity of first electrodes, which have a holding region for holding probe molecules which can bind macromolecular biopolymers; and
a multiplicity of second electrodes, the first and second electrodes being arranged in an electrode array.

16. The biosensor chip as claimed in claim 15, further comprising:
a multiplicity of third electrodes, the second electrodes and the third electrodes being designed in such a manner that the reduction/oxidation process takes place as part of the reduction/oxidation recycling operation at the second electrodes and at the third electrodes.
